(19) United States
(12) Patent Application Publication
(10) Pub. No.: US 2005/0239787 A1
(43) Pub. Date: Oct. 27, 2005

(54) Novel maxi-k channel blockers, methods of use and process for making the same

(76) Inventors: Michael A. Goetz, Scotch Plains, NJ (US); Gregory J. Kaczorowski, Edison, NJ (US); Richard L. Monaghan, Morristown, NJ (US); William R. Strohl, Bridgewater, NJ (US); Jan S. Tkacz, Piscataway, NJ (US)

Correspondence Address: MERCK AND CO., INC, P O BOX 2000, RAHWAY, NJ, 07065-0907, US

(21) Appl. No.: 10/511,664

(22) PCT Filed: Jun. 13, 2003
(86) PCT No.: PCT/US03/19013

§ 371 Date: Oct. 18, 2004

(30) Foreign Application Priority Data
Jun. 17, 2002 (US) 60389205

(51) Int. Cl. A61K 31/498; A61K 31/407; A61K 31/404

(52) U.S. Cl. 514/249; 514/410; 514/414; 514/415

(57) ABSTRACT

This invention relates to the use of potent potassium channel blockers or a formulation thereof in the treatment of glaucoma and other conditions related to elevated intraocular pressure in the eye of a patient. This invention also relates to the use of such compounds to provide a neuroprotective effect to the eye of a mammalian species, particularly humans.

This case claims the benefit of provisional application U.S. Ser. No. 60/389205, filed Jun. 17, 2002.

### BACKGROUND OF THE INVENTION

Glaucoma is a degenerative disease of the eye wherein the intraocular pressure is too high to permit normal eye function. Damage eventually occurs to the optic nerve head, resulting in irreversible loss of visual function. If untreated, glaucoma may eventually lead to blindness. Elevated intraocular pressure or ocular hypertension, is now believed by the majority of ophthalmologists to represent the earliest phase in the onset of glaucoma.

Many of the drugs formerly used to treat glaucoma proved unsatisfactory. The early methods of treating glaucoma employed pilocarpine and produced undesirable local effects that made this drug, though valuable, unsatisfactory as a first line drug. More recently, clinicians have noted that many β-adrenergic antagonists are effective in reducing intraocular pressure. While many of these agents are effective for this purpose, there exist some patients with whom this treatment is not effective or not sufficiently effective. Many of these agents also have other characteristics, e.g., membrane stabilizing activity, that become more apparent with increased doses and render them unacceptable for chronic ocular use and can also cause cardiovascular effects.

Although pilocarpine and β-adrenergic antagonists reduce intraocular pressure, none of these drugs manifests its action by inhibiting the enzyme carbonic anhydrase, and thus they do not take advantage of reducing the contribution to aqueous humor formation made by the carbonic anhydrase pathway.

Agents referred to as carbonic anhydrase inhibitors decrease the formation of aqueous humor by inhibiting the enzyme carbonic anhydrase. While such carbonic anhydrase inhibitors are now used to treat intraocular pressure by systemic and topical routes, current therapies using these agents, particularly those using systemic routes are still not without undesirable effects. Because carbonic anhydrase inhibitors have a profound effect in altering basic physiological processes, the avoidance of a systemic route of administration serves to diminish, if not entirely eliminate, those side effects caused by inhibition of carbonic anhydrase such as metabolic acidosis, vomiting, numbness, tingling, general malaise and the like. Topically effective carbonic anhydrase inhibitors are disclosed in U.S. Pat. Nos. 4,386,098; 4,416,890; 4,426,388; 4,668,697; 4,863,922; 4,797,413; 5,378,703, 5,240,923 and 5,153,192.

Prostaglandins and prostaglandin derivatives are also known to lower intraocular pressure. U.S. Pat. No. 4,883,819 to Bito describes the use and synthesis of PGAs, PGBs and PGCs in reducing intraocular pressure. U.S. Pat. No. 4,824,857 to Goh et al. describes the use and synthesis of PGD2 and derivatives thereof in lowering intraocular pressure including derivatives wherein C-10 is replaced with nitrogen. U.S. Pat. No. 5,001,153 to Ueno et al. describes the use and synthesis of 13,14-dihydro-15-keto prostaglandins and prostaglandin derivatives to lower intraocular pressure.

U.S. Pat. No. 4,599,353 describes the use of eicosanoids and eicosanoid derivatives including prostaglandins and prostaglandin inhibitors in lowering intraocular pressure.

Prostaglandin and prostaglandin derivatives lower intraocular pressure by increasing uveoscleral outflow. This is true for both the F type and A type of Pgs and hence presumably also for the B, C, D, E and J types of prostaglandins and derivatives thereof. A problem with using prostaglandin derivatives to lower intraocular pressure is that these compounds often induce an initial increase in intraocular pressure, can change the color of eye pigmentation and cause proliferation of some tissues surrounding the eye.

As can be seen, there are several current therapies for treating glaucoma and elevated intraocular pressure, but the efficacy and the side effect profiles of these agents are not ideal. Recently potassium channel blockers were found to reduce intraocular pressure in the eye and therefore provide yet one more approach to the treatment of ocular hypertension and the degenerative ocular conditions related thereto. Blockage of potassium channels can diminish fluid secretion, and under some circumstances, increase smooth muscle contraction and would be expected to lower IOP and have neuroprotective effects in the eye. (see U.S. Pat. Nos. 5,573,758 and 5,925,342; Moore, et al., Invest. Ophthalmol. Vis. Sci 38, 1997; WO 89/10757, WO94/28900, and WO 96/33719).

### SUMMARY OF THE INVENTION

This invention relates to the use of the compounds in Table 1 below:

TABLE 1

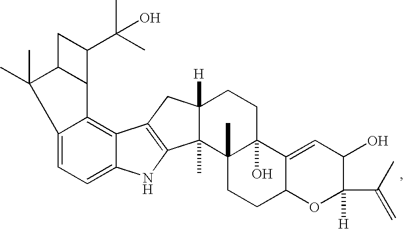

PC-M4

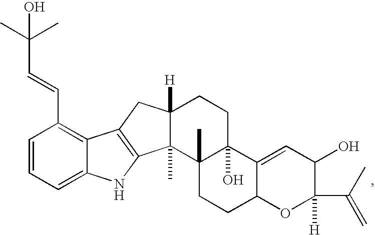

PC-M5

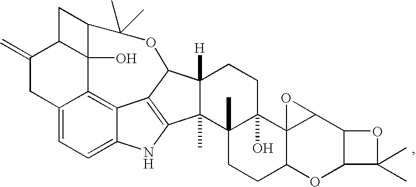

pennigritrem

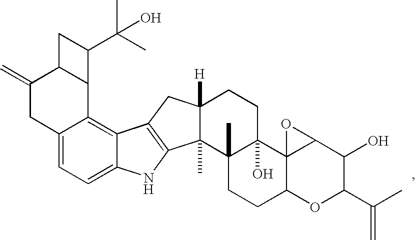

Secopenitrem B

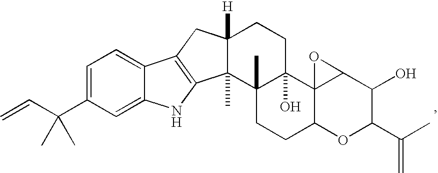

Sulpinine A

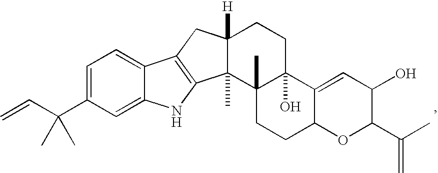

Sulpinine B

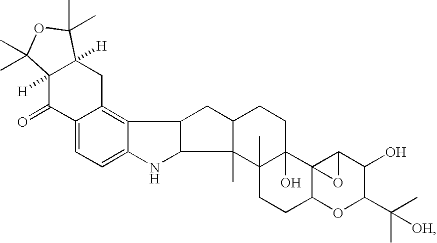

Lolitrem N

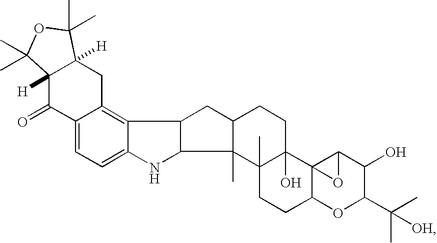

Lolitrem N, 31-epimer

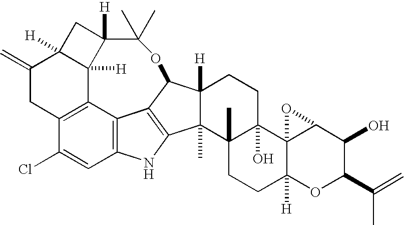

penitrem F

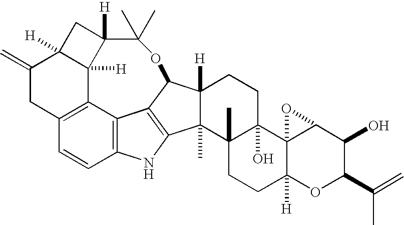

penitrem B

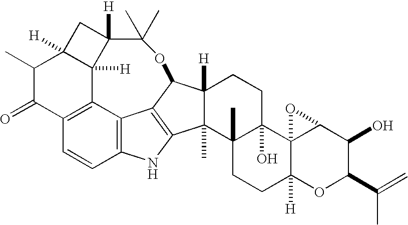

10-oxo, 11,33-dihydro, 15-deoxy, dechloro penitrem A

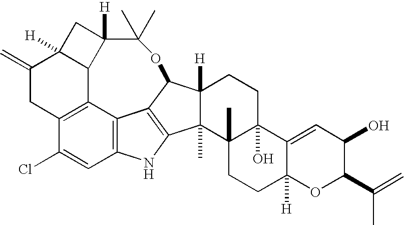

penitrem C

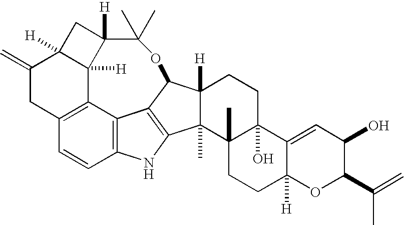

penitrem D

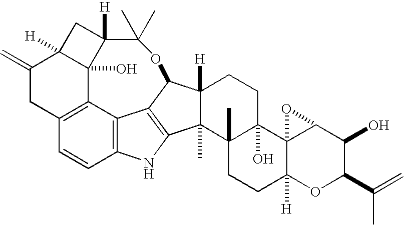

penitrem E

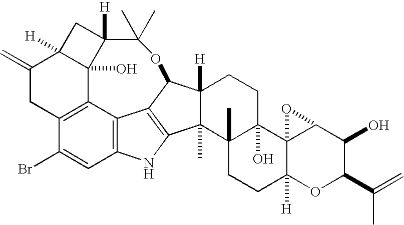

6-bromo-penitrem D

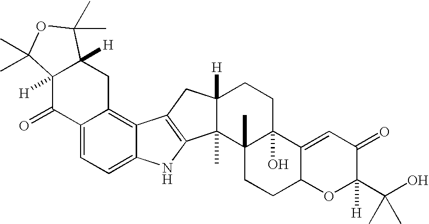

Loilline

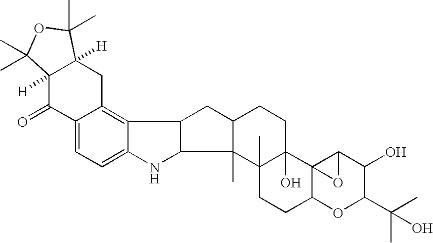

Lolitriol

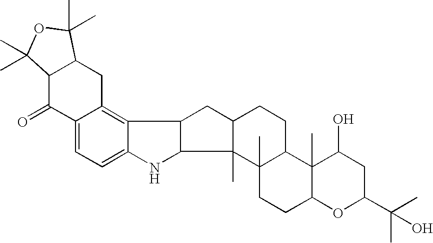

Lolicine A

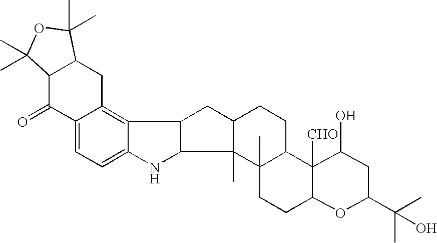

Lolicine B

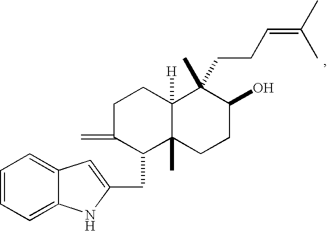

emindole DA

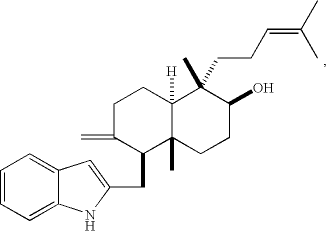

epi-emindole DA

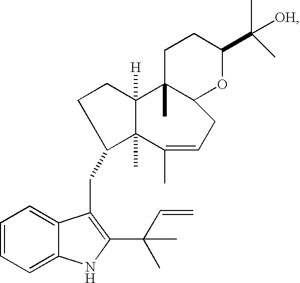

emindole PA

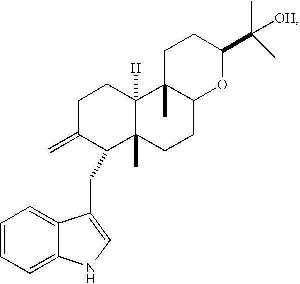

emindole DB

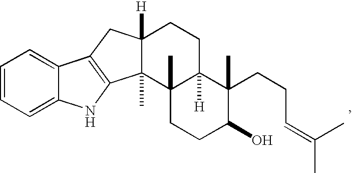

emindole SB

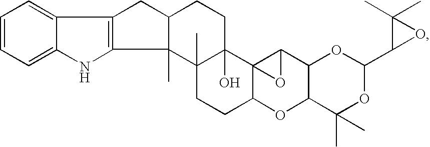

terpendole A

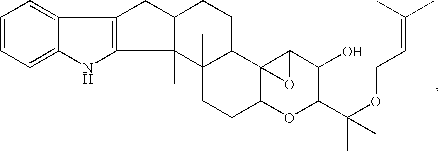

terpendole B

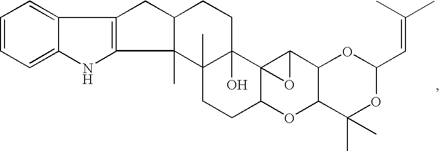

terpendole C

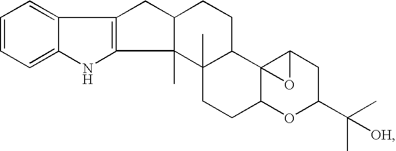

terpendole D

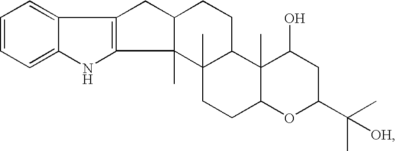

Terpendole E

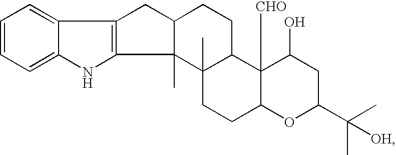

Terpendole G

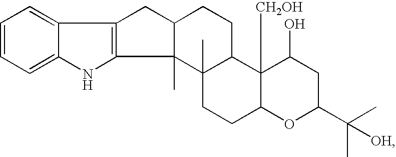

Terpendole F

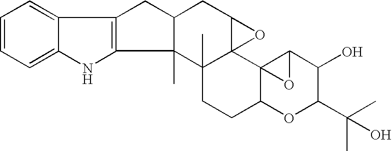

Terpendole H

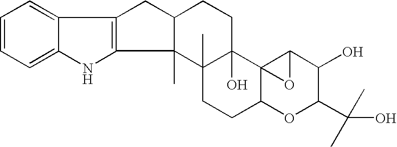

Terpendole I

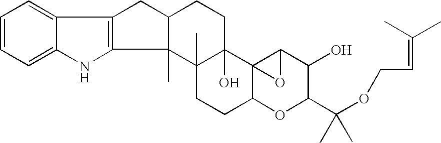

Terpendole J

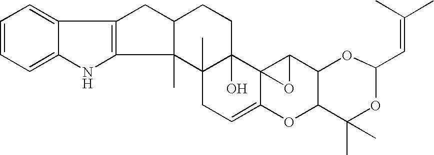

Terpendole K

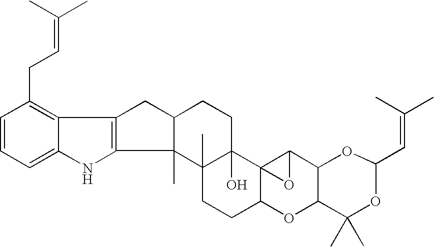

Terpendole L

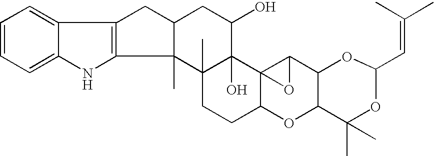

Terpendole M

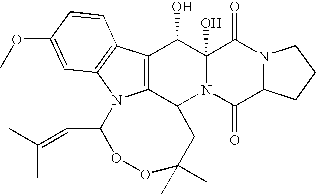

verruculogen

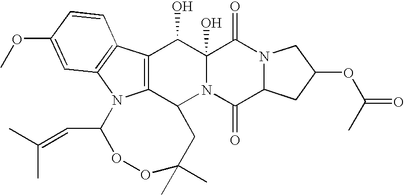

8-acetoxy-verruculogen

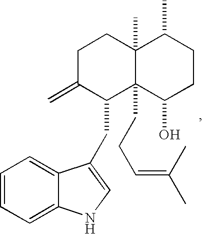

nominine

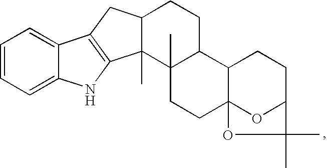

paspalicine

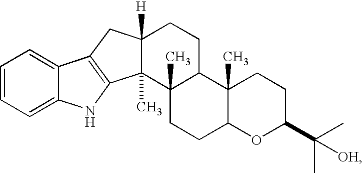

paspaline

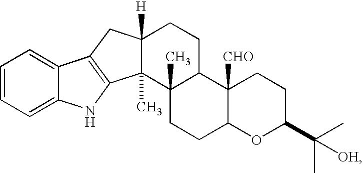

paspaline B

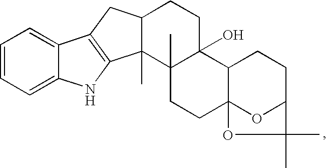

paspalinine

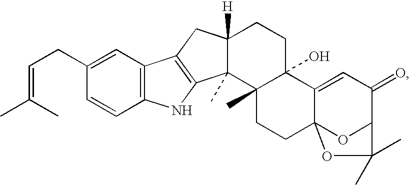

Paspalitrem A

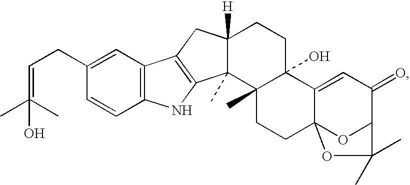

Paspalitrem B

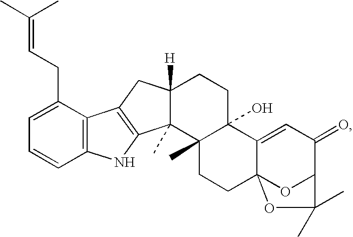

Paspalitrem C

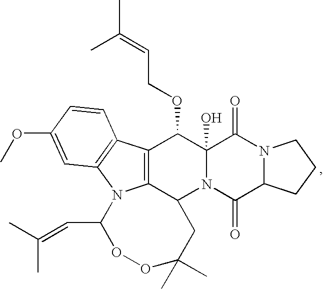

Fumitremorgen A

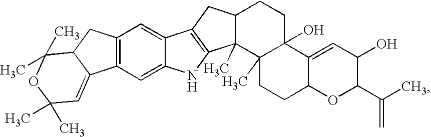

Janthitrem B

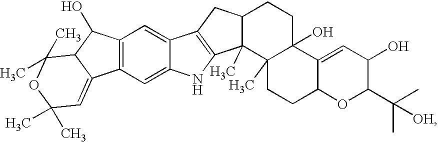

Janthitrem C

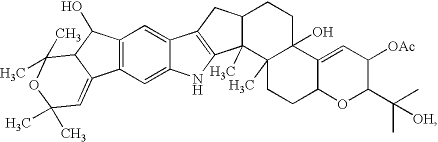

Janthitrem E

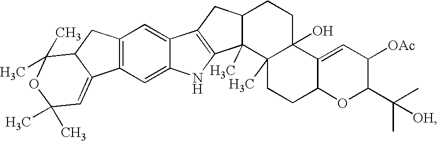

Janthitrem F

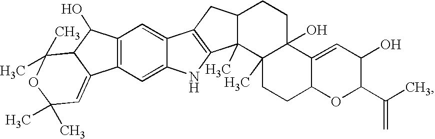

Janthitrem G

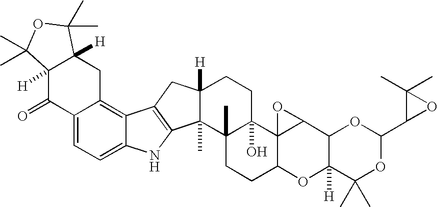

Lolitrem A

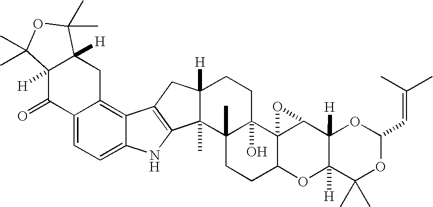

Lolitrem B

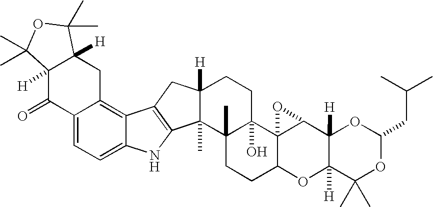

Lolitrem C

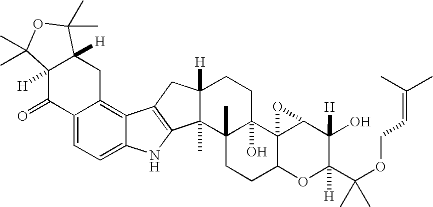

Lolitrem E

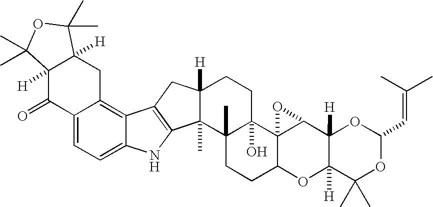

Lolitrem F

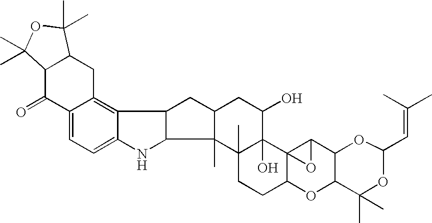

Lolitrem H

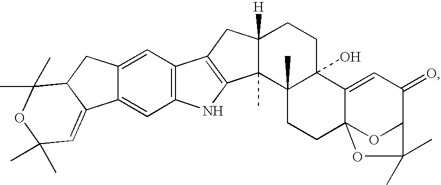

shearinine A

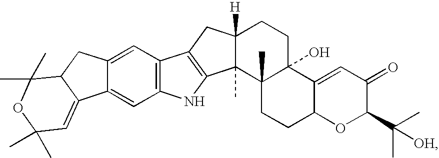

shearinine B

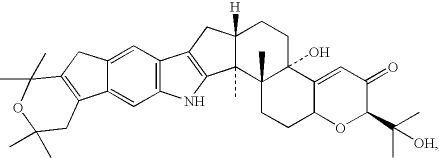

shearinine B isomer

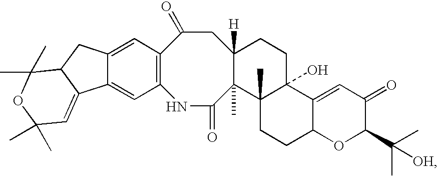

shearinine C

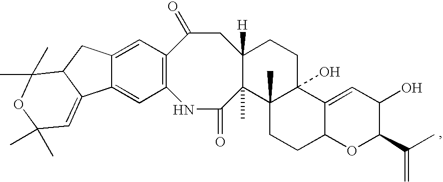

Shearinine C, 1′-deoxy, 1′,2′-didehydRo,3-beta alcohol

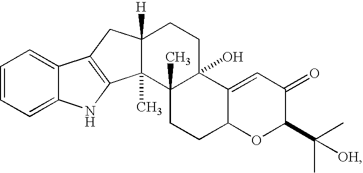

paxilline

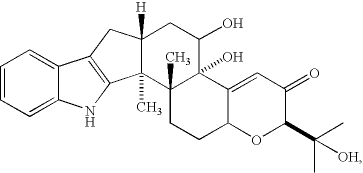

paxilline, 14-hydroxy

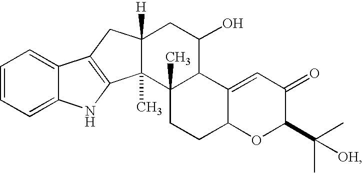

paxilline, 14-hydroxy, 4b-deoxy

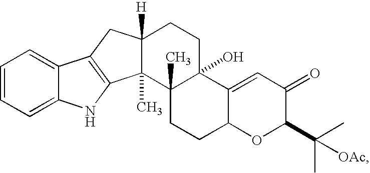

paxilline, 1-acetyl

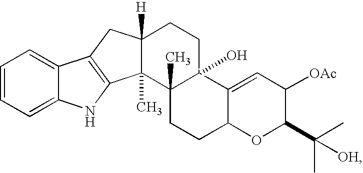

paxilline, 3-acetyl

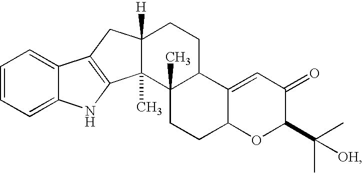

4b-deoxypaxilline

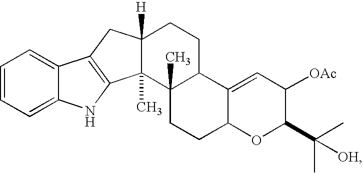

4b-deoxypaxilline, 3-acetyl

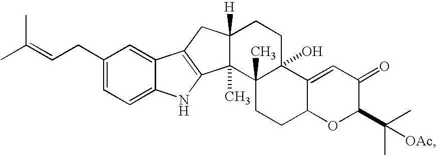

9-prenylpaxilline

or a pharmaceutically acceptable salt, enantiomer, diastereomer, tautomer or mixture thereof, as potent potassium channel blockers in the treatment of glaucoma and other conditions which are related to elevated intraocular pressure in the eye of a patient. Also encompassed by this invention is the use of such compounds to provide a neuroprotective effect to the eye of mammalian species, particularly humans. More particularly this invention relates to the treatment of glaucoma and ocular hypertension (elevated intraocular pressure) using the indole diterpene compounds mentioned above.

This and other aspects of the invention will be realized upon review of the specification as a whole.

### DETAILED DESCRIPTION OF THE INVENTION

In addition, the compounds disclosed herein may exist as tautomers and both tautomeric forms are intended to be encompassed by the scope of the invention, even though only one tautomeric structure is depicted. For example, any claim to compound A below is understood to include tautomeric structure B, and vice versa, as well as mixtures thereof.

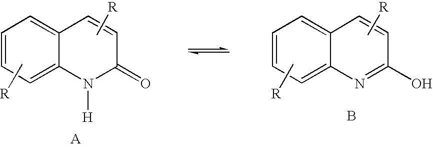

Also included within the scope of this invention are pharmaceutically acceptable salts or esters, where a basic or acidic group is present in the compounds listed above.

An embodiment of this invention is a method for treating ocular hypertension and/or glaucoma which comprises administering to a patient in need of such treatment a therapeutically effective amount of a compound of Table 1 above.

Another embodiment contemplates the method described above wherein the compounds are applied as a topical formulation.

Yet another embodiment contemplates the method described above wherein the topical formulation is a solution or suspension.

And yet another embodiment is the method described above, which comprises administering a second active ingredient, concurrently or consecutively, wherein the second active ingredient is an ocular hypotensive agent selected from a β-adrenergic blocking agent, adrenergic, agonist, a parasympathomimetic agent, a carbonic anhydrase inhibitor, EP4 agonist as disclosed in U.S. Ser. No. 60/386,641, filed Jun. 6, 2002 (Attorney Docket MC059PV), 60/421,402, filed Oct. 25,2002 (Attorney Docket MC067PV), 60/457,700, filed Mar. 26, 2003 (Attorney Docket MC080PV), 60/406,530, filed Aug. 28, 2002 (Attorney Docket MC060PV) and PCT applications PCT 02/38039, filed Nov. 27, 2002 and PCT 02/38040, filed Nov. 27, 2002, all incorporated by reference in its entirety herein, and a prostaglandin or a prostaglandin derivative.

Another embodiment is the method described above wherein the β-adrenergic blocking agent is timolol, levobunolol, carteolol, optipranolol, metapranolol or betaxolol; the parasympathomimetic agent is pilocarpine, carbachol, or phospholine iodide; adrenergic agonist is iopidine, brimonidine, epinephrine, or dipivephrin, the carbonic anhydrase inhibitor is dorzolamide, acetazolamide, metazolamide or brinzolamide; the prostaglandin is latanoprost or rescula, and the prostaglandin derivative is a hypotensive lipid derived from PGF2α prostaglandins.

A further embodiment is a method for treating macular edema or macular degeneration which comprises administering to a patient in need of such treatment a pharmaceutically effective amount of a compound in Table 1 above.

Another embodiment is the method described above wherein the compound of Table 1 is applied as a topical formulation.

Still another embodiment of this invention comprises administering a second active ingredient, concurrently or consecutively, wherein the second active ingredient is an ocular hypotensive agent selected from a β-adrenergic blocking agent, adrenergic agonist, a parasympathomimetic agent, a carbonic anhydrase inhibitor, and a prostaglandin or a prostaglandin derivative.

Another embodiment is the method described above wherein the β-adrenergic blocking agent is timolol, levobunolol, carteolol, optipranolol, metapranolol or betaxolol; the parasympathomimetic agent is pilocarpine, carbachol, or phospholine iodide; adrenergic agonist is iopidine, brimonidine, epinephrine, or dipivephrin, the carbonic anhydrase inhibitor is dorzolamide, acetazolamide, metazolamide or brinzolamide; the prostaglandin is latanoprost or rescula, and the prostaglandin derivative is a hypotensive lipid derived from PGF2α prostaglandins.

A further embodiment is illustrated by a method for increasing retinal and optic nerve head blood velocity or increasing retinal and optic nerve oxygen tension which comprises administering to a patient in need of such treatment a therapeutically effective amount of a compound of Table 1 above.

And another embodiment is the method described above wherein the compound of Table 1 is applied as a topical formulation.

Still another embodiment comprises administering a second active ingredient, concurrently or consecutively, wherein the second active ingredient is an ocular hypotensive agent selected from a β-adrenergic blocking agent, adrenergic agonist, a parasympathomimetic agent, a carbonic anhydrase inhibitor, and a prostaglandin or a prostaglandin derivative.

Another embodiment is the method described above wherein the β-adrenergic blocking agent is timolol, levobunolol, carteolol, optipranolol, metapranolol or betaxolol; the parasympathomimetic agent is pilocarpine, carbachol, or phospholine iodide; adrenergic agonist is iopidine, brimonidine, epinephrine, or dipivephrin, the carbonic anhydrase inhibitor is dorzolamide, acetazolamide, metazolamide or brinzolamide; the prostaglandin is latanoprost or rescula, and the prostaglandin derivative is a hypotensive lipid derived from PGF2α prostaglandins.

Another embodiment of the invention is a method for providing a neuroprotective effect to a mammalian eye which comprises administering to a patient in need of such treatment a therapeutically effective amount of a compound of Table 1 above.

Also within the scope of the invention is the method described above wherein the compound of Table 1 is applied as a topical formulation.

Still another embodiment comprises administering a second active ingredient, concurrently or consecutively, wherein the second active ingredient is an ocular hypotensive agent selected from a β-adrenergic blocking agent, adrenergic agonist, a parasympathomimetic agent, a carbonic anhydrase inhibitor, and a prostaglandin or a prostaglandin derivative.

Another embodiment is the method described above wherein the β-adrenergic blocking agent is timolol, levobunolol, carteolol, optipranolol, metapranolol or betaxolol; the parasympathomimetic agent is pilocarpine, carbachol, or phospholine iodide; adrenergic agonist is iopidine, brimonidine, epinephrine, or dipivephrin, the carbonic anhydrase inhibitor is dorzolamide, acetazolamide, metazolamide or brinzolamide; the prostaglandin is latanoprost or rescula, and the prostaglandin derivative is a hypotensive lipid derived from PGF2α prostaglandins.

Also contemplated to be within the scope of the present invention is a topical formulation of a compound in Table 1 as described above wherein the topical formulation also contains xanthan gum or gellan gum.

The invention is described herein in detail using the terms defined below unless otherwise specified.

Also included within the scope of this invention are pharmaceutically acceptable salts or esters, where a basic or acidic group is present in a compound of Table 1.

This invention is also concerned with a method of treating ocular hypertension or glaucoma by administering to a patient in need thereof one of the compounds of Table 1 in combination with an ocular hypotensive agent selected from a β-adrenergic blocking agent such as timolol, optipranolol, levobunolol, metapranolol, carteolol, betaxalol and the like, a parasympathomimetic agent such as pilocarpine, carbachol, phospholine iodide, and the like, adrenergic agonist such as iopidine, brimodine, epinephrine, dipivephrin, and the like, carbonic anhydrase inhibitor such as dorzolamide, acetazolamide, metazolamide or brinzolamide, a prostaglandin such as latanoprost, rescula, S1033 or a prostaglandin derivative such as a hypotensive lipid derived from PGF2α a prostaglandins. An example of a hypotensive lipid (the carboxylic acid group on the α-chain link of the basic prostaglandin structure is replaced with electrochemically neutral substituents) is that in which the carboxylic acid group is replaced with a C₁₋₆ alkoxy group such as OCH₃ (PGF₂ₐ1-OCH₃), or a hydroxy group (PGF₂ₐ1-OH).

Preferred potassium channel blockers are calcium activated potassium channel blockers. More preferred potassium channel blockers are high conductance, calcium activated potassium (maxi-K) channel blockers. Maxi-K channels are a family of ion channels that are prevalent in neuronal, smooth muscle and epithelial tissues and which are gated by membrane potential and intracellular Ca²⁺.

Intraocular pressure (IOP) is controlled by aqueous humor dynamics. Aqueous humor is produced at the level of the non-pigmented ciliary epithelium and is cleared primarily via outflow through the trabecular meshwork. Aqueous humor inflow is controlled by ion transport processes. It is thought that maxi-K channels in non-pigmented ciliary epithelial cells indirectly control chloride secretion by two mechanisms; these channels maintain a hyperpolarized membrane potential (interior negative) which provides a driving force for chloride efflux from the cell, and they also provide a counter ion (K⁺) for chloride ion movement. Water moves passively with KCl allowing production of aqueous humor. Inhibition of maxi-K channels in this tissue would diminish inflow. Maxi-K channels have also been shown to control the contractility of certain smooth muscle tissues, and, in some cases, channel blockers can contract quiescent muscle, or increase the myogenic activity of spontaneously active tissue. Contraction of ciliary muscle would open the trabecular meshwork and stimulate aqueous humor outflow, as occurs with pilocarpine. Therefore maxi-K channels could profoundly influence aqueous humor dynamics in several ways; blocking this channel would decrease IOP by affecting inflow or outflow processes or by a combination of affecting both inflow/outflow processes.

The present invention is based upon the finding that maxi-K channels, if blocked, inhibit aqueous humor production by inhibiting net solute and H₂O efflux and therefore lower IOP. This finding suggests that maxi-K channel blockers are useful for treating other ophthamological dysfunctions such as macular edema and macular degeneration. It is known that lowering of IOP promotes increased blood flow to the retina and optic nerve. Accordingly, this invention relates to a method for treating macular edema, macular degeneration or a combination thereof.

Additionally, macular edema is swelling within the retina within the critically important central visual zone at the posterior pole of the eye. An accumulation of fluid within the retina tends to detach the neural elements from one another and from their local blood supply, creating a dormancy of visual function in the area.

Glaucoma is characterized by progressive atrophy of the optic nerve and is frequently associated with elevated intraocular pressure (IOP). It is possible to treat glaucoma, however, without necessarily affecting IOP by using drugs that impart a neuroprotective effect. See Arch. Ophthalmol. Vol. 112, Jan 1994, pp. 37-44; Investigative Ophthamol. & Visual Science, 32, 5, Apr. 1991, pp. 1593-99. It is believed that maxi-K channel blockers which lower IOP are useful for providing a neuroprotective effect. They are also believed to be effective for increasing retinal and optic nerve head blood velocity and increasing retinal and optic nerve oxygen by lowering IOP, which when coupled together benefits optic nerve health. As a result, this invention further relates to a method for increasing retinal and optic nerve head blood velocity, increasing retinal and optic nerve oxygen tension as well as providing a neuroprotective effect or a combination thereof.

The maxi-K channel blockers used are preferably administered in the form of ophthalmic pharmaceutical compositions adapted for topical administration to the eye such as solutions, ointments, creams or as a solid insert. Formulations of this compound may contain from 0.01 to 5% and especially 0.5 to 2% of medicament. Higher dosages as, for example, about 10% or lower dosages can be employed provided the dose is effective in reducing intraocular pressure, treating glaucoma, increasing blood flow velocity or oxygen tension or providing a neuroprotective effect. For a single dose, from between 0.001 to 5.0 mg, preferably 0.005 to 2.0 mg, and especially 0.005 to 1.0 mg of the compound can be applied to the human eye.

The pharmaceutical preparation which contains the compound may be conveniently admixed with a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, polyalkylene glycols, petroleum based jelly, ethyl cellulose, ethyl oleate, carboxymethyl-cellulose, polyvinylpyrrolidone, isopropyl myristate and other conventionally employed acceptable carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like. The pharmaceutical preparation may also be in the form of a microparticle formulation. The pharmaceutical preparation may also be in the form of a solid insert. For example, one may use a solid water soluble polymer as the carrier for the medicament. The polymer used to form the insert may be any water soluble non-toxic polymer, for example, cellulose derivatives such as methylcellulose, sodium carboxymethyl cellulose, (hydroxyloweralkyl cellulose), hydroxyethyl cellulose, hydroxypropyl cellulose, hydroxypropylmethyl cellulose; acrylates such as polyacrylic acid salts, ethylacrylates, polyactylamides; natural products such as gelatin, alginates, pectins, tragacanth, karaya, chondrus, agar, acacia; the starch derivatives such as starch acetate, hydroxymethyl starch ethers, hydroxypropyl starch, as well as other synthetic derivatives such as polyvinyl alcohol, polyvinyl pyrrolidone, polyvinyl methyl ether, polyethylene oxide, neutralized carbopol and xanthan gum, gellan gum, and mixtures of said polymer.

Suitable subjects for the administration of the formulation of the present invention include primates, man and other animals, particularly man and domesticated animals such as cats and dogs.

The pharmaceutical preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The ophthalmic solution or suspension may be administered as often as necessary to maintain an acceptable IOP level in the eye. It is contemplated that administration to the mammalian eye will be about once or twice daily.

For topical ocular administration the novel formulations of this invention may take the form of solutions, gels, ointments, suspensions or solid inserts, formulated so that a unit dosage comprises a therapeutically effective amount of the active component or some multiple thereof in the case of a combination therapy.

The maxi-K channel blocker of the compounds of Table 1 are known and are commercially available or can be prepared as described in references 1-32 cited in Schedules A and B below and which are incorporated herein by reference in their entirety.

### Schedule A

1. B. J. Wilson, Science, 1964, 144, 177
2. M. R. TePaske, J. Nat. Prod., 1992, 55, 1080
3. K. Nozawa, Chem Comm., 1987, 1157
4. K. Nozawa, J. C. S. Perkin I, 1988, 1689 and 2155
5. K. Kawai, J. C. S. Perkin I, 1994, 1673
6. J. B. Gloer, J. Org Chem, 1989, 54, 2530
7. K. Nozawa, J. C. S. Perkin I, 1988, 2607
8. a) M. Yamazaki, Chem Pharm Bull, 1980, 28, 245 ; b) M. Uramoto, Heterocycles, 1982, 17, 349 ; c) J. B. Day, J. Gen Microbiol., 1980, 117, 405
9. A. E. De Jesus, J. C. S. Perkin I, 1984, 697
10. S. C. Mundayfinch, J. Agric. Food Chem, 1997, 45, 199 and 204
11. S. C. Mundayfinch, J. Agric. Food Chem, 1998, 46, 590
12. a) C. O. Miles, J. Agric. Food Chem, 1994, 42, 1488 ; b) S. C. Mundayfinch, J. Agric. Food Chem, 1995, 43, 1283 c) S. C. Mundayfinch, J. Agric. Food Chem, 1996, 44, 2782; d) R. T. Gallagher, Chem Comm., 1984, 614
13. a) T. Fehr, Helv Chim Acta, 1966, 49, 1907 ; b) R. J. Cole, J. Agric Food Chem, 1977, 25, 826 ; c) R. J. Cole, J. Agric Food Chem, 1977, 25, 1197 ; c) R. J. Cole, J. Agric Food Chem, 1981, 29, 293 d) K. Nozawa, Chem Pharm Bull, 1989, 37, 626
14. T. Fehr, Helv Chim Acta, 1966, 49, 1907
15. S. C. Mundayfinch, Phytochemistry, 1996, 41, 327
16. R. J. Cole, J. Agric Food Chem, 1977, 25, 826 and 1197
17. R. J. Cole, Can J. Microbiol, 1974, 20, 1159
18. K. Nozawa, J. C. S. Perkin I, 1988, 2607
19. K. Nozawa, Chem Pharm Bull, 1989,37, 1387
20. P. G. Mantle, Phytochemistry, 1994, 36, 1209
21. T. Hosoe, Chem Pharm Bull, 1990, 38, 3473
22. G. N. Belofsky, Tetrahedron, 1995, 51, 3959
23. a) B. J. Wilson, Nature, 1968, 220, 77 ; b) A. E. De Jesus, J. C. S. Perkin I, 1983, 1847, 1857 and 1863
24. J. A. Laakso, J. Agric. Food Chem, 1993, 41, 973
25. H. Hayashi, Chem Express, 1993, 8, 233
26. A. E. De Jesus, J. C. S. Perkin I, 1983, 1847 and 1857
27. T. Yamaguchi, Phytochemistry, 1993, 32, 1177
28. J. Penn, J C S Perkin I, 1992, 23
29. J. A. Laakso, J. Org Chem, 1992, 57, 2066
30. H. Tomoda, J. Antibiotics, 1995, 48, 793
31. X-H. Huang, J. Antibiotics, 1995, 48, 1 and 5

32. W. A. Gatenby, J. Agric Food Chem, 1999, 47, 1092

Schedule B

| reference | compound | source |
|---|---|---|
| 1 | Aflatrem | Aspergillus flavus |
| 2 | Aflatrem, beta | Aspergillus flavus |
| 3 | Emindole DA | Emericella striata |
| 4 | Emindole DA, 5 epimer | Emericella striata |
| 5 | Emindole PA | Emericella purpurea |
| 6 | Emindole DB | Emericella purpurea |
| 7 | Emindole SB | Emericella purpurea |
| 8 | Fumitremorgen A | Aspergillus fumigatus |
| 9 | Janthitrem B | Penicillium janthinellum |
| 9 | Janthitrem C | Penicillium janthinellum |
| 9 | Janthitrem E | Penicillium janthinellum |
| 9 | Janthitrem F | Penicillium janthinellum |
| 9 | Janthitrem G | Penicillium janthinellum |
| 10 | Lolilline | ryegrass infected with |
| | | Neotyphodium lolii |
| 11 | Lolitriol | ryegrass infected with |
| | | Neotyphodium lolii |
| 11 | Lolicine A | ryegrass infected with |
| | | Neotyphodium lolii |
| 11 | Lolicine B | ryegrass infected with |
| | | Neotyphodium lolii |
| 12 | Lolitrem A | ryegrass infected with |
| | | Neotyphodium lolii |
| 12 | Lolitrem B | ryegrass infected with |
| | | Neotyphodium lolii |
| 12 | Lolitrem C | ryegrass infected with |
| | | Neotyphodium lolii |
| 12 | Lolitrem E | ryegrass infected with |
| | | Neotyphodium lolii |
| 12 | Lolitrem F | ryegrass infected with |
| | | Neotyphodium lolii |
| 32 | Lolitrem H | ryegrass infected with |
| | | Neotyphodium lolii |
| 11 | Lolitrem N | ryegrass infected with |
| | | Neotyphodium lolii |
| 11 | Lolitrem N, 31 epimer | ryegrass infected with |
| | | Neotyphodium lolii |
| 6 | Nominine | Aspergillus nomius |
| 13 | Paspalicine | Claviceps paspali |
| 14 | Paspaline | Claviceps paspali |
| 15 | Paspaline B | Penicillium paxilli |
| 13 | Paspalinine | Aspergillus flavus |
| 16 | Paspalitrem A | Claviceps paspali |
| 16 | Paspalitrem B | Claviceps paspali |
| 13 | Paspalitrem C | Claviceps paspali |
| 17 | Paxilline | Emericella striata |
| 20 | Paxilline, 14-alpha-hydroxy | Penicillium paxilli |
| 20 | Paxilline, 14-alpha-hydroxy, | Penicillium paxilli |
| | 4b-deoxy | |
| 19 | Paxilline, 1-acetyl | Emericella striata |
| 21 | Paxilline, 3beta-alcohol, | Penicillium crustosum |
| | 3-acetyl | |
| 18 | Paxilline, 4b-deoxy | Emericella striata |
| 21 | Paxilline, 4b-deoxy, 3-acetyl | Penicillium paxilli |
| 22 | Paxilline, 9-prenyl | Eupenicillium shearii |
| 23 | Penitrem A | Penicillium crustosum |
| 23 | Penitrem F | Penicillium crustosum |
| 23 | Penitrem B | Penicillium crustosum |
| 24 | Penitrem A, 6-dechloro, 15- | Aspergillus sulphureus |
| | deoxy, 10-oxo, 11,33-dihydro | |
| 26 | Penitrem C | Penicillium crustosum |
| 26 | Penitrem D | Penicillium crustosum |
| 23 | Penitrem E | Penicillium crustosum |
| 25 | Penitrem E, 6-bromo | Penicillium simplicissimum |
| 27 | PC-M4 | Penicillium crustosum |
| 27 | PC-M5 | Penicillium crustosum |
| 28 | Pennitigrem | Penicillium nigricans |
| 29 | Secopenitrem B | Aspergillus sulphureus |
| 22 | Shearinine A | Eupenicillium shearii |
| 22 | Shearinine B | Eupenicillium shearii |
| 22 | Shearinine B, isomer | Eupenicillium shearii |
| 22 | Shearinine C | Eupenicillium shearii |
| 22 | Shearinine C, 1′-deoxy, 1′,2′- | Penicillium sp. |
| | didehydro, 3-beta alcohol | |
| 29 | Sulpinine B | Aspergillus sulphureus |
| 29 | Sulpinine A | Aspergillus sulphureus |
| 31 | Terpendole A | Albophoma |
| | | yamanashiensis |
| 31 | Terpendole B | Albophoma |
| | | yamanashiensis |
| 31 | Terpendole C | Albophoma |
| | | yamanashiensis |
| 31 | Terpendole D | Albophoma |
| | | yamanashiensis |
| 30 | Terpendole E | Albophoma |
| | | yamanashiensis |
| 30 | Terpendole F | Albophoma |
| | | yamanashiensis |
| 30 | Terpendole G | Albophoma |
| | | yamanashiensis |
| 30 | Terpendole H | Albophoma |
| | | yamanashiensis |
| 30 | Terpendole I | Albophoma |
| | | yamanashiensis |
| 30 | Terpendole J | Albophoma |
| | | yamanashiensis |
| 30 | Terpendole K | Albophoma |
| | | yamanashiensis |
| 30 | Terpendole L | Albophoma |
| | | yamanashiensis |
| 32 | Terpendole M | ryegrass infected with |
| | | Neotyphodium lolii |
| 8 | Verruculogen | Aspergillus fumigatus |
| 8 | Verruculogen, 8-beta acetoxy | Penicillium verruculosum |

The following examples are used to exemplify the invention, but should not be construed so as to limit the scope of the invention.

### EXAMPLE 1

### Electrophysiological Assays of Compound Effects on High-Conductance Calcium-Activated Potassium Channels

Methods:

Patch clamp recordings of currents flowing through high-conductance calcium-activated potassium (Maxi-K) channels were made from membrane patches excised from CHO cells constitutively expressing the α-subunit of the Maxi-K channel or HEK293 cells constitutively expressing both α- and β1-subunits using conventional techniques (Hamill et al., 1981, Pfluügers Archiv. 391, 85-100) at room temperature. Glass capillary tubing (Garner #7052) was pulled in two stages to yield micropipettes with tip diameters of approximately 1-2 microns. Pipettes were typically filled with solutions containing (mM): 150 KCl, 10 Hepes (4-(2-hydroxyethyl)-1-piperazine methanesulfonic acid), 1 Mg, 0.01 Ca, and adjusted to pH 7.20 with 3.7 mM KOH. After forming a high resistance (>10⁹ ohms) seal between the plasma membrane and the pipette, the pipette was withdrawn from the cell, forming an excised inside-out membrane patch. The patch was excised into a bath solution containing (mM): 150 KCl, 10 Hepes, 5 EGTA (ethylene glycol bis(β-aminoethyl ether)-N,N,N′,N′-tetraacetic acid), sufficient Ca to yield a free Ca concentration of 1-5 μM, and the pH was adjusted to 7.2 with KOH. For example, 4.193 mM Ca was added to give a free concentration of 1 μM at 22° C. An EPC9 amplifier (HEKA Elektronic, Lambrect, Germany) was used to control the voltage and to measure the currents flowing across the membrane patch. The input to the headstage was connected to the pipette solution with a Ag/AgCl wire, and the amplifier ground was connected to the bath solution with a Ag/AgCl wire covered with a tube filled with agar dissolved in 0.2 M KCl. The identity of Maxi-K currents was confirmed by the sensitivity of channel open probability to membrane potential and intracellular calcium concentration.

Data acquisition was controlled by PULSE software (HEKA Elektronic) and stored on the hard drive of a MacIntosh computer (Apple Computers) for later analysis using PULSEFIT (HEKA Elektronic) and Igor (Wavemetrics, Oswego, Oreg.) software.

Results:

The effects of the compounds of the present invention on Maxi-K channels were examined in excised inside-out membrane patches. The membrane potential was held at −80 mV and brief voltage steps to positive membrane potentials (typically +50 mV) were applied once per 15 seconds to transiently open Maxi-K channels. The fraction of channels blocked in each experiment was calculated from the reduction in peak current caused by application of the specified compound to the internal side of the membrane patch. As a positive control in each experiment, Maxi-K currents were eliminated at pulse potentials after the patch was transiently exposed to a low concentration of calcium (<10 nM) made by adding 1 mM EGTA to the standard bath solution with no added calcium. The activity for blocking Maxi-K channel currents by the compounds of Table 1 is 100 nM or less.

### EXAMPLE 2

### The Activity of the Compound can also be Quantified by the Following Assay

The identification of inhibitors of the Maxi-K channel is based on the ability of expressed Maxi-K channels to set cellular resting potential after transfection of both alpha and beta1 subunits of the channel in HEK-293 cells and after being incubated with potassium channel blockers that selectively eliminate the endogenous potassium conductances of HEK-293 cells. In the absence of Maxi-K channel inhibitors, the transfected HBEK-293 cells display a hyperpolarized membrane potential, negative inside, close to E_{K} (−80 mV) which is a consequence of the activity of Maxi-K channels. Blockade of the Maxi-K channel by incubation with Maxi-K channel blockers will cause cell depolarization. Changes in membrane potential can be determined with voltage-sensitive fluorescence resonance energy transfer FRET) dye pairs that use two components, a donor coumarin (CC₂DMPE) and an acceptor oxanol (DiSBAC₂(3)).

Oxanol is a lipophilic anion and distributes across the membrane according to membrane potential. Under normal conditions, when the inside of the cell is negative with respect to the outside, oxanol is accumulated at the outer leaflet of the membrane and excitation of coumarin will cause FRET to occur. Conditions that lead to membrane depolarization will cause the oxanol to redistribute to the inside of the cell, and, as a consequence, a decrease in FRET. Thus, the ratio change (donor/acceptor) increases after membrane depolarization, which determines if a test compound actively blocks the maxi-K channel.

The HEK-293 cells were obtained from American Type Culture Collection, 12301 Parklawn Drive, Rockville, Md., 20852 under accession number ATCC CRL-1573. Any restrictions relating to public access to the cell line shall be irrevocably removed upon patent issuance.

Transfection of the alpha and betal subunits of the maxi-K channel in HEK-293 cells was carried out as follows: HEK-293 cells were plated in 100 mm tissue culture treated dishes at a density of 3×10⁶ cells per dish, and a total of five dishes were prepared. Cells were grown in a medium consisting of Dulbecco's Modified Eagle Medium (DMEM) supplemented with 10% Fetal Bovine serum, 1× L-Glutamine, and 1× Penicillin/Streptomycin, at 37° C., 10% CO₂. For transfection with Maxi-K hα(pCIneo) and Maxi-K hβ1(pIRESpuro) DNAs, 150 μl FuGENE6™ was added drop-wise into 10 ml of serum free/phenol-red free DMEM and allowed to incubate at room temperature for 5 minutes. Then, the FuGENE6™ solution was added drop-wise to a DNA solution containing 25 μg of each plasmid DNA, and incubated at room temperature for 30 minutes. After the incubation period, 2 ml of the FuGENE6™/DNA solution was added drop-wise to each plate of cells and the cells were allowed to grow two days under the same conditions as described above. At the end of the second day, cells were put under selection media that consisted of DMEM supplemented with both 600 μg/ml G418 and 0.75 μg/ml puromycin. Cells were grown until separate colonies were formed. Five colonies were collected and transferred to a 6 well tissue culture treated dish. A total of 75 colonies were collected. Cells were allowed to grow until a confluent monolayer was obtained. Cells were then tested for the presence of Maxi-K channel alpha and betal subunits using an assay that monitors binding of ¹²⁵I-iberiotoxin-D19Y/Y36F to the channel. Cells expressing ¹²⁵I-iberiotoxin-D19Y/Y36F binding activity were then evaluated in a functional assay that monitors the capability of Maxi-K channels to control the membrane potential of transfected HEK-293 cells using fluorescence resonance energy transfer (FREI) Aurora Biosciences technology with a VIPR instrument. The colony giving the largest signal to noise ratio was subjected to limiting dilution. For this, cells were resuspended at approximately 5 cells/ml, and 200 μl were plated in individual wells in a 96 well tissue culture treated plate, to add ca. one cell per well. A total of two 96 well plates were made. When a confluent monolayer was formed, the cells were transferred to 6 well tissue culture treated plates. A total of 62 wells were transferred. When a confluent monolayer was obtained, cells were tested using the FRET-functional assay. Transfected cells giving the best signal to noise ratio were identified and used in subsequent functional assays.

For Functional Assays:

The transfected cells (2E+06 Cells/mL) are then plated on 96-well poly-D-lysine plates at a density of about 100,000 cells/well and incubated for about 16 to about 24 hours. The medium is aspirated of the cells and the cells washed one time with 100 μl of Dulbecco's phosphate buffered saline (D-PBS). One hundred microliters of about 9 μM coumarin (CC₂DMPE)-0.02% pluronic-127 in D-PBS per well is added and the wells are incubated in the dark for about 30 minutes. The cells are washed two times with 100 μl of Dulbecco's phosphate-buffered saline and 100 μl of about 4.5 μM of oxanol (DiSBAC₂(3)) in (mM) 140 NaCl, 0.1 KCl, 2 CaCl₂, 1 MgCl₂, 20 Hepes-NaOH, pH 7.4, 10 glucose is added. Three micromolar of an inhibitor of endogenous potassium conductance of HEK-293 cells is added. A maxi-K channel blocker is added (about 0.01 micromolar to about 10 micromolar) and the cells are incubated at room temperature in the dark for about 30 minutes.

The plates are loaded into a voltage/ion probe reader (VIPR) instrument, and the fluorescence emission of both CC₂DMPE and DiSBAC₂(3) are recorded for 10 sec. At this point, 100 μl of high-potassium solution (mM): 140 KCl, 2 CaCl₂, 1 MgCl₂, 20 Hepes-KOH, pH 7.4, 10 glucose are added and the fluorescence emission of both dyes recorded for an additional 10 sec. The ratio CC₂DMPE/DiSBAC₂(3), before addition of high-potassium solution equals 1. In the absence of maxi-K channel inhibitor, the ratio after addition of high-potassium solution varies between 1.65-2.0. When the maxi-K channel has been completely inhibited by either a known standard or test compound, this ratio remains at 1. It is possible, therefore, to titrate the activity of a maxi-K channel inhibitor by monitoring the concentration-dependent change in the fluorescence ratio. The activity for blocking maxi-K channels by compounds of Table 1 is 1 μM or less.

### EXAMPLE 3

### Intraocular Pressure (IOP) Measurements in Rabbits

Normotensive Dutch Belted rabbits (2.3 kg) of either sex are maintained on a 12-hour light/dark cycle during these experiments. Intraocular pressure (IOP) is measured using a calibrated pneumatonometer (Alcon Applanation Pneumatonograph), and results are expressed in millimeters of mercury (mmHg). Before tonometry, one drop of 0.05% proparacaine is applied to the corneas to minimize any discomfort to the animal. Two base-line (control) readings are taken at (−0.5 and 0 hr.) after which Compounds of Table 1 are administered topically (unilaterally applied into the conjunctival sac) in a 25 μl volume with the contralateral (fellow) eye receiving an equal volume of vehicle. A masked design is utilized, where the person involved in drug administration and measurement of IOP have no knowledge of the solutions' contents. Subsequently, IOP measurements are taken at 0.5, 1, 2, 3, 4, 5 and 6 hr after topical applications of drug. At the end of each day's measurements, stability of the tonometer was determined using the calibrator/verifier.

1. A method for treating ocular hypertension or glaucoma which comprises administering to a patient in need of such treatment a therapeutically effective amount of a compound of Table 1:
TABLE 1 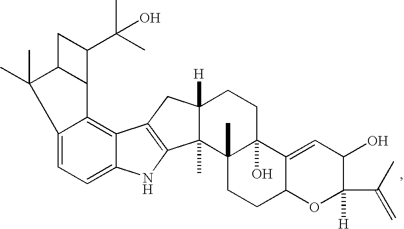
PC-M4 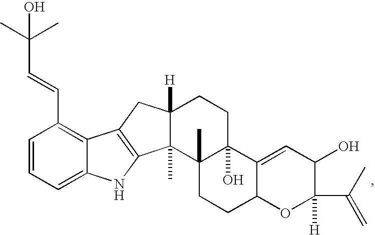
PC-M5 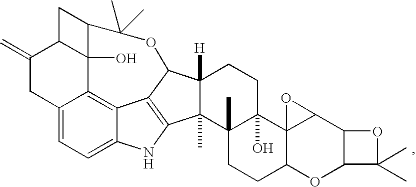
pennigritrem 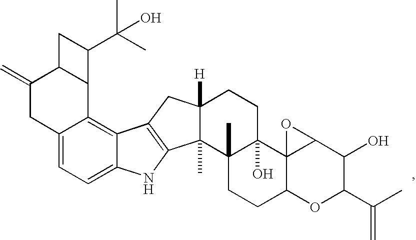
Secopenitrem B 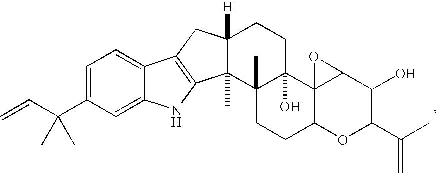
Sulpinine A 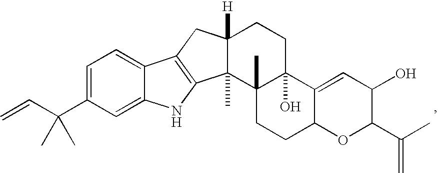
Sulpinine B 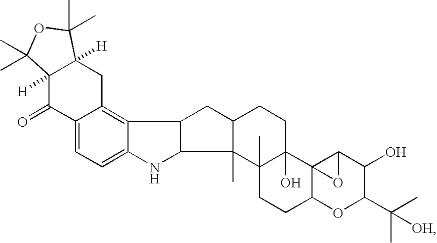
Lolitrem N 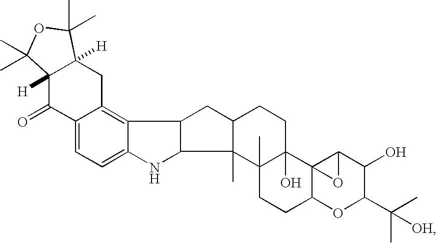
Lolitrem N, 31-epimer 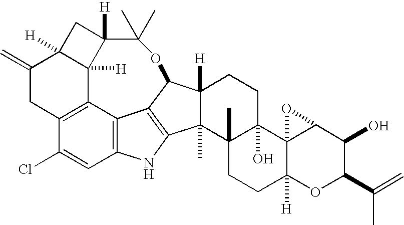
penitrem F 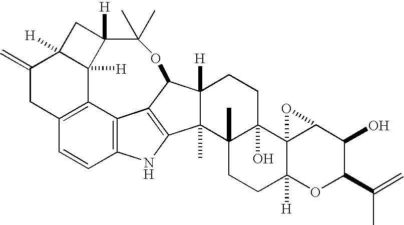
penitrem B 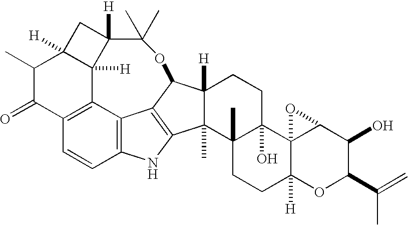
10-oxo, 11,33-dihydro, 15-deoxy, dechloro penitrem A 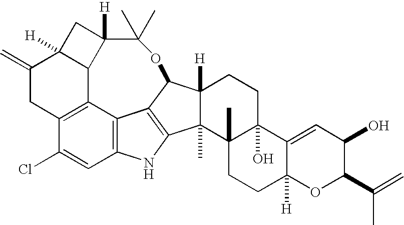
penitrem C 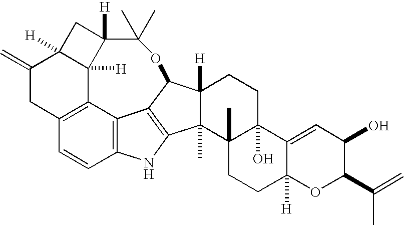
penitrem D 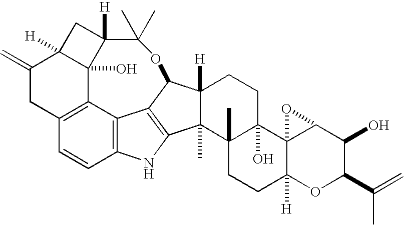
penitrem E 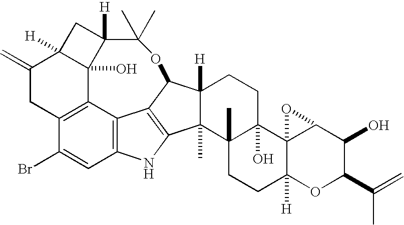
6-bromo-penitrem D 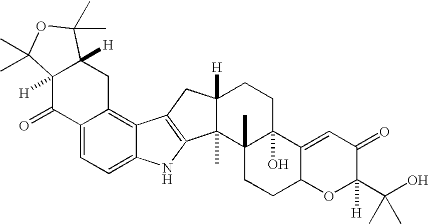
Loilline 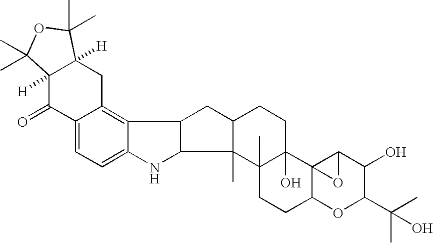
Lolitriol 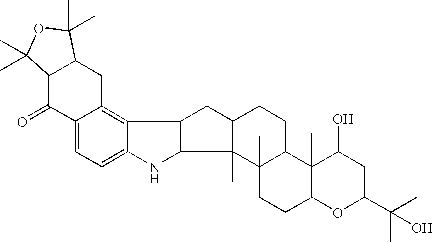
Lolicine A 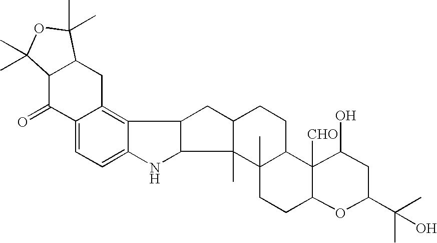
Lolicine B 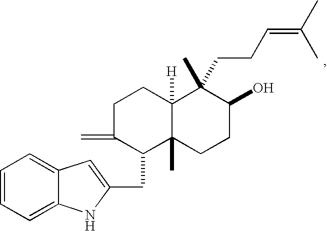
emindole DA 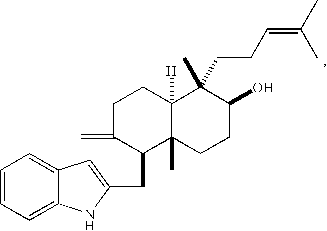
epi-emindole DA 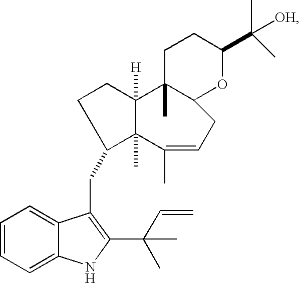
emindole PA 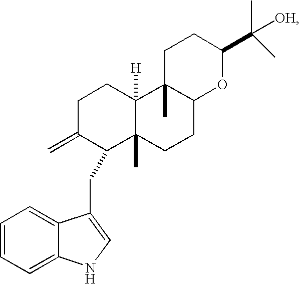
emindole DB 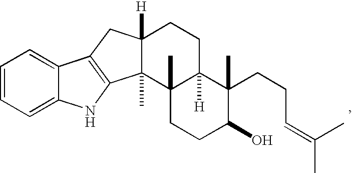
emindole SB 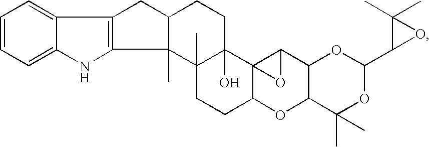
terpendole A 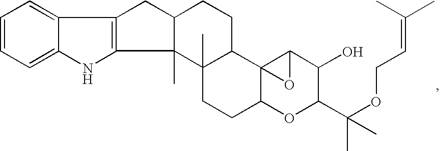
terpendole B 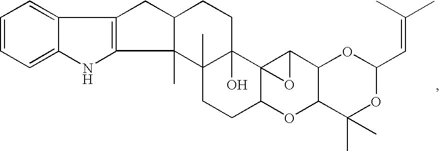
terpendole C 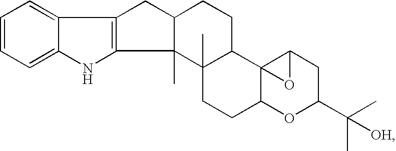
terpendole D 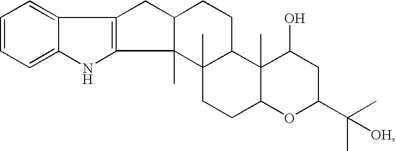
Terpendole E 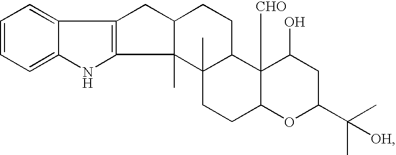
Terpendole G 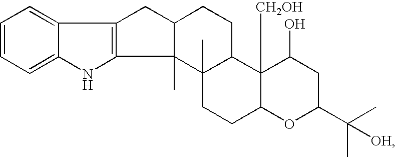
Terpendole F 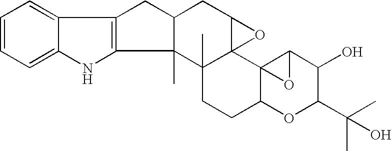
Terpendole H 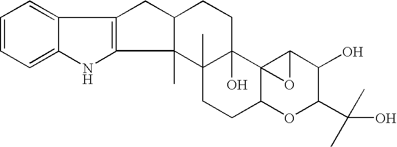
Terpendole I 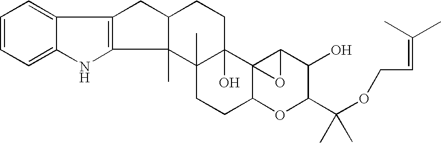
Terpendole J 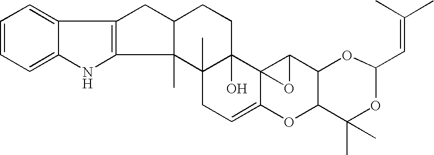
Terpendole K 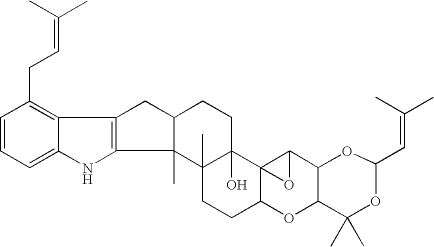
Terpendole L 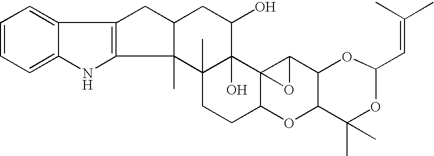
Terpendole M 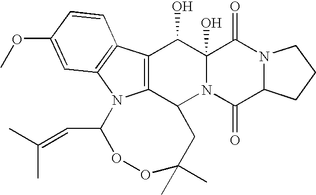
verruculogen 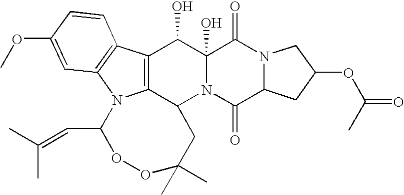
8-acetoxy-verruculogen 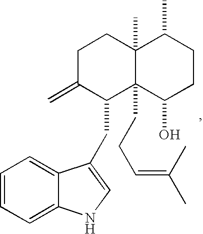
nominine 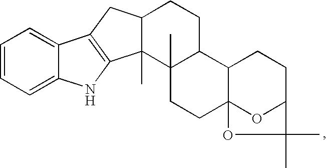
paspalicine 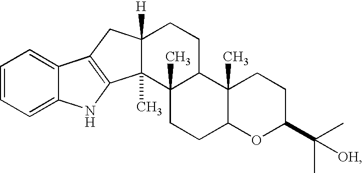
paspaline 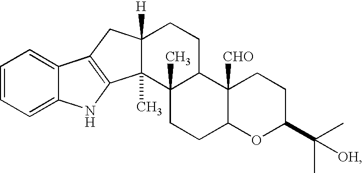
paspaline B 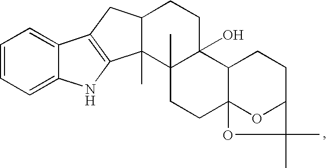
paspalinine 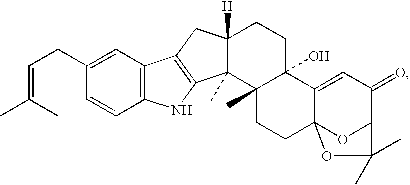
Paspalitrem A 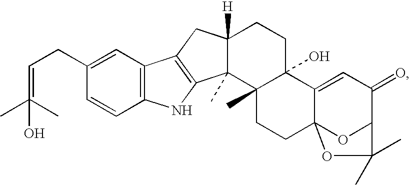
Paspalitrem B 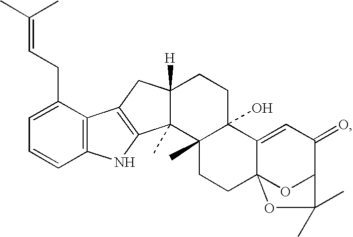
Paspalitrem C 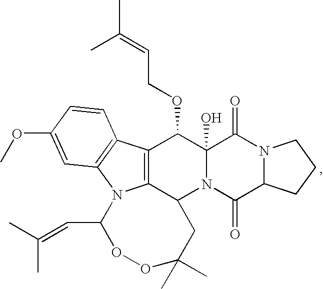
Fumitremorgen A 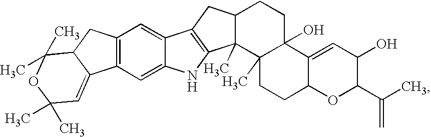
Janthitrem B 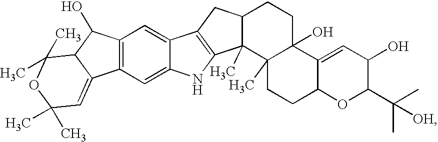
Janthitrem C 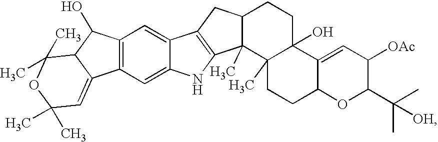
Janthitrem E 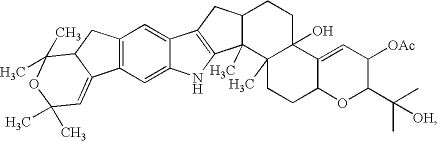
Janthitrem F 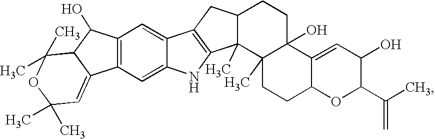
Janthitrem G 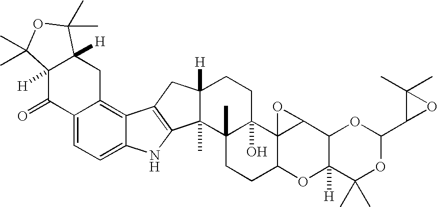
Lolitrem A 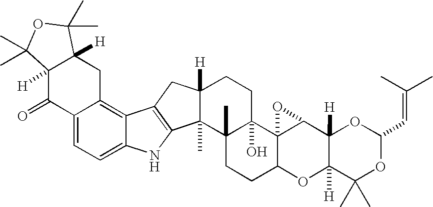
Lolitrem B 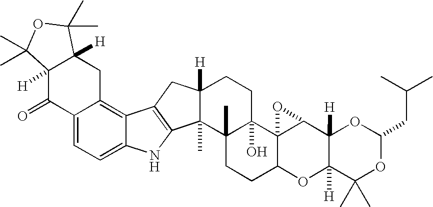
Lolitrem C 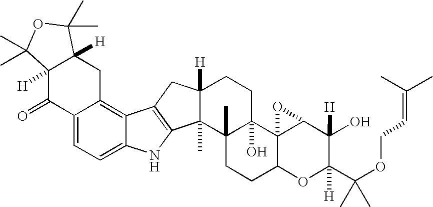
Lolitrem E 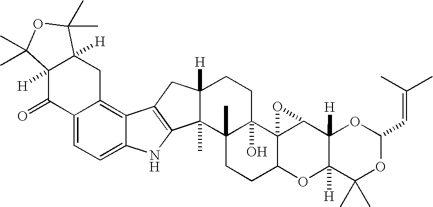
Lolitrem F 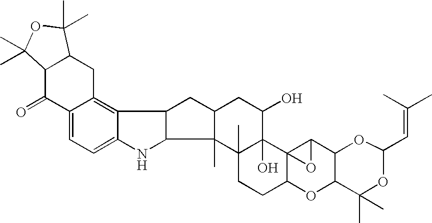
Lolitrem H 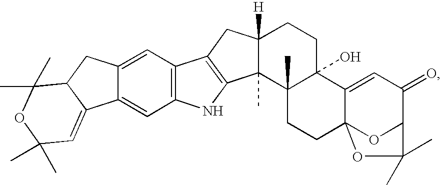
shearinine A 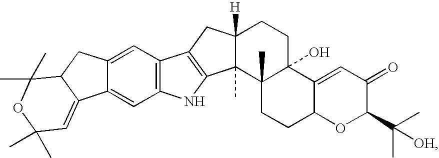
shearinine B 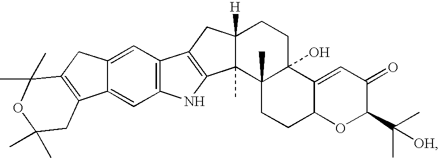
shearinine B isomer 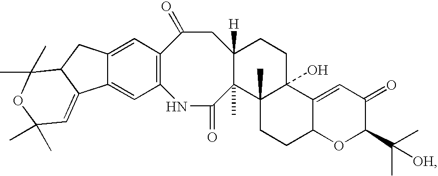
shearinine C 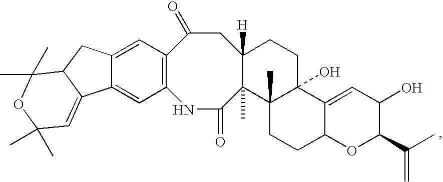
Shearinine C, 1′-deoxy, 1′,2′-didehydRo,3-beta alcohol 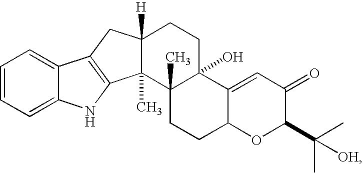
paxilline 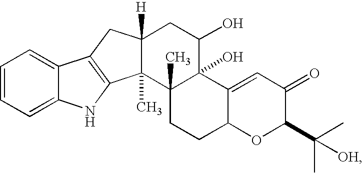
paxilline, 14-hydroxy 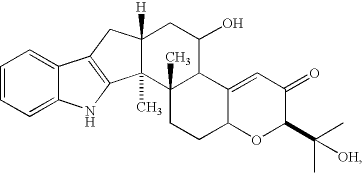
paxilline, 14-hydroxy, 4b-deoxy 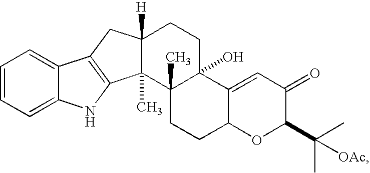
paxilline, 1-acetyl 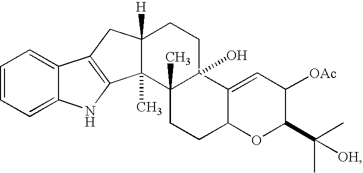
paxilline, 3-acetyl 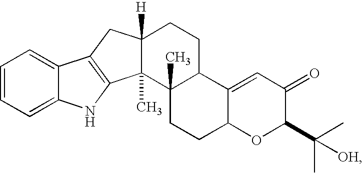
4b-deoxypaxilline 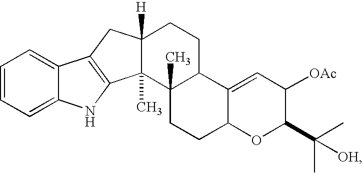
4b-deoxypaxilline, 3-acetyl 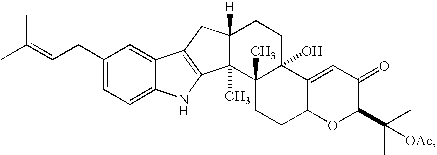
9-prenylpaxilline
or a pharmaceutically acceptable salt, enantiomer, diastereomer, tautomer or mixture thereof.

2. The method according to claim 1 wherein the compound of formula I is applied as a topical formulation.

3. The method according to claim 3 wherein the topical formulation is a solution or suspension.

4. The method of claim 3, which comprises administering a second active ingredient, concurrently or consecutively, wherein the second active ingredient is a hypotensive agent selected from a β-adrenergic blocking agent, adrenergic agonist, a parasympathomimetic agent, a carbonic anhydrase inhibitor, EP4 agonist and a prostaglandin or a prostaglandin derivative.

5. The method according to claim 4 wherein the β-adrenergic blocking agent is timolol, levobunolol, carteolol, optipranolol, metapranolol or betaxolol; the parasympathomimetic agent is pilocarpine, carbachol, or phospholine iodide; adrenergic agonist is iopidine, brimonidine, epinephrine, or dipivephrin, the carbonic anhydrase inhibitor is dorzolamide, acetazolamide, metazolamide or brinzolamide; the prostaglandin is latanoprost or rescula, and the prostaglandin derivative is a hypotensive lipid derived from PGF2α prostaglandins.

6. A method according to claim 2 in which the topical formulation contains xanthan gum or gellan gum.

7. A method for treating macular edema, macular degeneration, for providing a neuroprotective effect, increasing retinal and optic nerve head blood velocity or increasing retinal and optic nerve oxygen tension which comprises administering to a patient in need of such treatment a pharmaceutically effective amount of a compound as recited in claim 1

8. The method according to claim 7 wherein the compound of formula I is applied as a topical formulation in the form of a solution or suspension.

9. The method of claim 8, which comprises administering a second active ingredient, concurrently or consecutively, wherein the second active ingredient is a hypotensive agent selected from a β-adrenergic blocking agent, adrenergic agonist, a parasympathomimetic agent, a carbonic anhydrase inhibitor, EP4 agonist and a prostaglandin or a prostaglandin derivative.

10. The method according to claim 9 wherein the β-adrenergic blocking agent is timolol, levobunolol, carteolol, optipranolol, metapranolol or betaxolol; the parasympathomimetic agent is pilocarpine, carbachol, or phospholine iodide; adrenergic agonist is iopidine, brimonidine, epinephrine, or dipivephrin, the carbonic anhydrase inhibitor is dorzolamide, acetazolamide, metazolamide or brinzolamide; the prostaglandin is latanoprost or rescula, and the prostaglandin derivative is a hypotensive lipid derived from PGF2α prostaglandins.

11. A method according to claim 8 in which the topical formulation contains xanthan gum or gellan gum.
